SEQUENCE LISTING (3 sequences)

SEQ ID NO: 1 - DNA, 191 bp, Artificial Sequence
  individual total DNA sequence information - partial example
atggggttgagtgtgtagtgatggggttgagtgtgtagtgatggggttgagtgtgtagtgatggggttgagtgtgtagtgatggggttgagtgtgtagtgatggggttgagtgtgtagtgatggggttgagtgtgtttttttaaaaaaaaaaaaaaaaaagcgccccccccccccccggggggggggttgt

SEQ ID NO: 2 - DNA, 17 bp, Artificial Sequence
  LD block representative DNA sequence
tggggttgagtgtgtag

SEQ ID NO: 3 - DNA, 11 bp, Artificial Sequence
  personal identification DNA sequence 1
agtgtgtagtg

(19) United States
(12) Patent Application Publication
(10) Pub. No.: US 2020/0160935 A1
(43) Pub. Date: May 21, 2020

(54) CLOUD-BASED GENE ANALYSIS SERVICE METHOD AND PLATFORM

(71) Applicant: ICHROGENE, INC., Suwon-si (KR)

(72) Inventors: Young Ah SHIN, Yongin-Si (KR); Seung Bum SEO, Gwangju-si (KR); Phil Sun SHIN, Yongin-si (KR)

(21) Appl. No.: 16/231,062

(22) Filed: Dec. 21, 2018

(30) Foreign Application Priority Data
Nov. 20, 2018 (KR) 10-2018-0143271

(51) Int. Cl. G16B 20/10; G16B 50/40; G16H 50/30; G06F 21/33

(52) U.S. Cl. CPC G16B 20/10; G06F 21/33; G16H 50/30; G16B 50/40

(57) ABSTRACT

A cloud-based gene analysis service platform includes: a communication module configured to communicate with a terminal connected to a laboratory; a controller configured to monitor generation of information on a genome corresponding to an individual through the communication module; and a storage unit configured to encrypt and store the generated information on the genome, wherein the controller performs analysis on the basis of the information on the genome and personal phenotypic information. Accordingly, it is possible to effectively protect gene information and provide individual customized service.

### BACKGROUND

### 1. Field

The present disclosure relates to a cloud computing-based gene analysis service method and platform.

### 2. Discussion of the Related Technology

The human body includes innumerable cells which have nuclei therein. Cell nuclei contain most of the cell's genetic material in the form of chromosomes. A chromosome within a nucleus is a deoxyribonucleic acid (DNA) molecule retaining the genetic information of a living organism. The DNA molecule comprises nucleotide chains, each consisting of four particular nucleobases adenine (A), guanine (G), cytosine (C), and thymine (T).

DNA is a hereditary carrier with which a living organism can transfer its own genetic information to the next generation. For sexual reproduction, offspring receives pairs of chromosomes from the male and female parents which are responsible for the chromosome pairs by halves, each. A human has a total of 23 pairs of chromosomes consisting of 22 pairs of autosomal chromosomes and one pair of sex chromosomes. A genome is the genetic material covering all chromosomes. Of the chromosomes, a region that codes for a particular function is termed a gene.

For humans, genome sequences are 99.9% identical among individuals, with a difference by 0.1% thereamong. The 0.1% base sequence difference is, in the most part, attributed to variation in a single base pair that occurs at a specific position (locus) in the genome. This feature at a specific locus is termed single nucleotide polymorphism (hereinafter referred to as “SNP”). From SNP frequencies in populations, differences in genetic distance, disease, characteristic, and trait can be analyzed between populations and between individuals.

As described above, not only can genome sequence information that differs from an individual to another intrinsically characterize individuals, but also various analyses can be performed on the basis of genome sequence information.

Therefore, a need exists for a method for producing and managing genome sequence information more safely.

Meanwhile, the aforementioned information is disclosed only as background information for helping in understanding the present disclosure. No decision and no opinion on whether any of the above content can be applied as the prior art of the present disclosure have been made yet.

### SUMMARY

An embodiment of the present disclosure proposes a cloud-based gene analysis service method.

The technical subjects pursued in the present disclosure may not be limited to the above mentioned technical subjects, and other technical subjects which are not mentioned may be clearly understood, through the following descriptions, by those skilled in the art of the present disclosure.

In accordance with an aspect of the present disclosure, a gene analysis service method performed by a processor is provided. The gene analysis service method includes: monitoring generation of information on a genome corresponding to an individual; encrypting and storing the generated information on the genome; and performing analysis on the basis of the information on the genome and personal phenotypic information.

More specifically, the gene analysis service method may further include authenticating at least one of one or more owners who provide genomes, one or more users who use information on the genomes of the owners, and one or more managers who manage a platform through a token method.

More specifically, the gene analysis service method may further include, when the users or the managers inquire about or use information on a genome of a particular owner, receiving consent from the particular owner.

More specifically, the monitoring of the generation may include monitoring a first step of extracting DNA from a genome provided from an owner, a second step of checking DNA concentration, and a third step of generating human mutant information through a gene experiment, the gene analysis service method further including decrypting, with a private key, human mutant information encrypted with a public key.

More specifically, the human mutant information may include at least one piece of Single Nucleotide Polymorphism (SNP) information, Short Indel information, and copy number variation information.

More specifically, the storing of the generated information may include encrypting and storing partial information generated by encrypting at least one piece of DNA sequence information which represents a genome-wide region, gender maker information, personal identification information, and DNA sequence information related to getting insurance, and whole personal DNA sequence information together.

More specifically, the performing of the analysis may include predicting personal disease risk on the basis of phenotypic information including at least one piece of information on a lifestyle, information on a disease history, and clinic information, and the information on the genome.

More specifically, the gene analysis service method may further include providing at least one piece of information on meals, information on exercise, information on health, and information on sleep to the individual in a personalized manner on the basis of the predicted personal disease risk.

More specifically, the predicting of the personal disease risk may include configuring the phenotypic information and the information on the genome in an input matrix and extracting features through a plurality of layers and expressing the personal disease risk in a binary number on the basis of a full connection scheme of the extracted features.

In accordance with another aspect of the present disclosure, a cloud-based gene analysis service platform is provided. The cloud-based gene analysis service platform includes: a communication module configured to communicate with a terminal connected to a laboratory; a controller configured to monitor generation of information on a genome corresponding to an individual through the communication module; and a storage unit configured to encrypt and store the generated information on the genome, wherein the controller performs analysis on the basis of the information on the genome and personal phenotypic information.

More specifically, the cloud-based gene analysis service platform may further include an authentication module configured to perform authentication in a token method, wherein the controller authenticates at least one of one or more owners who provide genomes, one or more users who use information on the genomes of the owners, and one or more managers who manage the platform through the authentication module.

More specifically, when the users or the managers inquire about or use information on a genome of a particular owner, the controller may receive consent from the particular owner through the communication module.

More specifically, the cloud-based gene analysis service platform may include a storage unit configured to store data, wherein the controller encrypts partial information generated by encrypting at least one piece of DNA sequence information which represents a genome-wide region, gender maker information, personal identification information, and DNA sequence information related to getting insurance, and whole personal DNA sequence information together and stores the encrypted information in the storage unit.

More specifically, the cloud-based gene analysis service platform may further include an analysis module configured to analyze gene information, wherein the controller controls the analysis module to predict personal disease risk on the basis of phenotypic information including at least one piece of information on a lifestyle, information on a disease history, and clinic information, and the information on the genome.

More specifically, the controller may provide at least one piece of information on meals, information on exercise, information on health, and information on sleep to the individual in a personalized manner on the basis of the predicted personal disease risk.

According to the present disclosure, the following effects are obtained.

First, it is possible to improve reliability of gene management by safely protecting sensitive gene information.

Second, it is possible to further reinforce security by encrypting again and storing feature information for identifying individuals among genome information.

Third, it is possible to provide a health care service for aging society by analyzing the correlation between phenotypic information and gene information.

Fourth, it is possible to more efficiently protect information by applying a verified cloud system.

Effects obtainable from the present disclosure may not be limited to the above mentioned effects, and other effects which are not mentioned may be clearly understood, through the following descriptions, by those skilled in the art of the present disclosure.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a system diagram schematically illustrating a cloud-based gene analysis service platform according to an embodiment of the present disclosure;

FIG. 2 is a block diagram illustrating the configuration of the cloud-based gene analysis service platform illustrated in FIG. 1; and

FIGS. 3 to 6 illustrate various driving of a gene analysis service platform according to an embodiment of the present disclosure.

### DETAILED DESCRIPTION OF EMBODIMENTS

Hereinafter, various embodiments of the present disclosure will be described in more detail with reference to the accompanying drawings. However, in description of the present disclosure, when it is determined that a detailed description of relevant known functions or configurations unnecessarily makes the main subject of the present disclosure unclear, the detailed description thereof will be omitted.

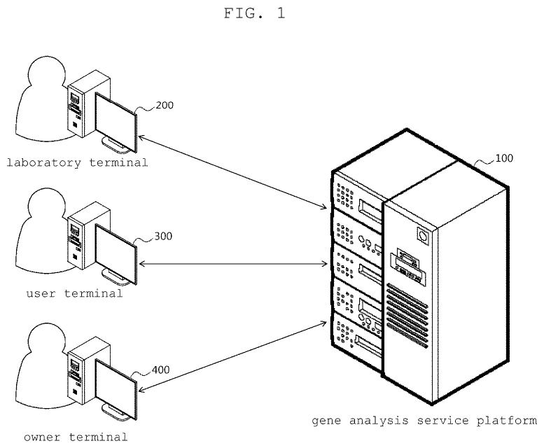

FIG. 1 is a system diagram schematically illustrating a cloud-based gene analysis service platform 100 according to an embodiment of the present disclosure.

The gene analysis service platform 100 is a system based on a cloud computing system beyond the existing typical system between a server and a client. Although it is illustrated that the number of gene analysis service platforms is one, the number thereof may include more entities than illustrated in the embodiment.

For information security, the gene analysis service platform 100 may complexly apply technologies such as a cloud-based micro service, a Relational Database Service (RDS), a Hardware Security Module (HSM), a JSON Web Token (JWT), a Secure Socket Layer (SSL), and encryption algorithms (RSA, ARIA, Encryption, GCM, and AES).

The gene analysis service platform 100 may collect gene information of various peoples from a laboratory terminal 200. For example, the gene analysis service platform 100 may receive, decrypt, and then process gene information encrypted for information security. The gene analysis service platform 100 may communicate with the laboratory terminal 200 through a Web Application Firewall and a Virtual Private Network (VPN) included in an API Gateway. That is, the laboratory terminal 200 may be considered as part of the gene analysis service platform 100.

The laboratory terminal 200 may decode and generate personal genome information, extract DNA, perform quantitative/qualitative analysis on the extracted DNA, and perform various experiments.

After performing a particular process, the laboratory terminal 200 may encrypt and provide information after the process to the gene analysis service platform 100.

A user terminal 300 is a terminal which desires to use personal gene information stored in the gene analysis service platform 100, and an owner terminal 400 may be a terminal corresponding to a terminal used by an owner who provides particular genome information or a terminal corresponding to an owner.

The gene analysis service platform 100 may issue tokens to a management terminal for managing the gene analysis service platform 100, the laboratory terminal 200, the user terminal 300, and the owner terminal 400. In embodiments, the tokens correspond to encryption required for receiving a particular service and may further include information on a barcode or a QR code.

The gene analysis service platform 100 may monitor generation of information on the genome corresponding to individuals. That is, the gene analysis service platform 100 may monitor gene information collected by the laboratory terminal 200 stage by stage.

Further, the gene analysis service platform 100 may encrypt and store the generated information on genome. The gene analysis service platform 100 may store information on the whole genome and may further encrypt and store information on a particular function included in the genome. The gene analysis service platform 100 may include a Network Access Server (NSA)-type storage unit to store various pieces of information but embodiments thereof are not limited thereto.

The gene analysis service platform 100 may perform various analyses on the basis of information on genome and phenotypic information of individuals and a detailed method thereof will be described below and is omitted herein.

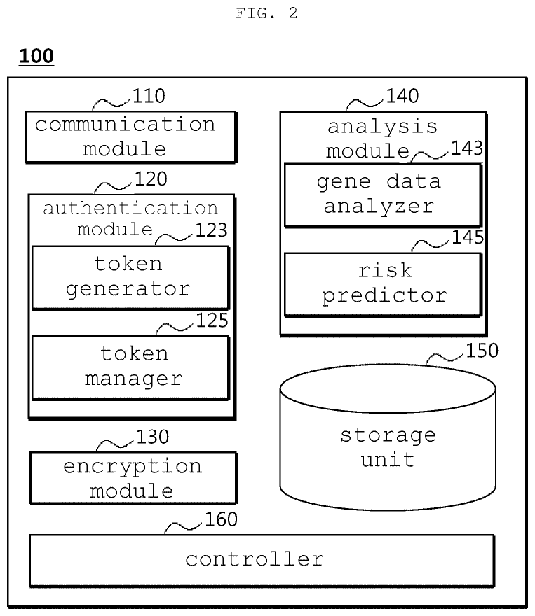

FIG. 2 is a block diagram illustrating the configuration of the gene analysis service platform 100.

Referring to FIG. 2, the gene analysis service platform 100 includes a communication module 110, an authentication module 120, an encryption module 130, an analysis module 140, a storage unit 150, and a controller 160. The elements illustrated in FIG. are not essential to implement the gene analysis service platform 100, so that the gene analysis service platform 100 described throughout the specification may have more or fewer elements than those listed above, and the controller 160 may overall control other elements.

The communication module 110 may communicate with external devices, such as the laboratory terminal, the user terminal, the management terminal, and the owner terminal. The communication module 110 may include a mobile communication module and a short-range communication module, and may further include any module for performing wired/wireless communication.

The authentication module 120 may authenticate at least one of one or more owners who provide genome, one or more users who use information on a genome of the owners, and managers who manage the platform through a token method.

The authentication module 120 includes a token generator 123 and a token manager 125, wherein the token generator 123 is a module configured to generate a token and the token manager 125 is a module configured to manage the generated token.

The encryption module 130 is a module configured to encrypt genome information, gene information, and personal identification information and may use Ron Shamir Adleman (RSA) corresponding to an encryption algorithm and Academy Research Institute Agency (ARIA) encryption corresponding to a block encryption algorithm, but embodiments thereof are not limited thereto.

The analysis module 140 is a module configured to analysis gene information and may perform various analyses on the basis of gene information and phenotypic information. The analysis module 140 includes a gene data analyzer 143 and a risk predictor 145.

The analysis module 140 may predict personal disease risk on the basis of phenotypic information including lifestyle information, information on a medical history, and clinical information, and gene information.

The storage unit 150 may store gene information and various pieces of information on individuals.

The controller 160 corresponds to a module configured to overall control the gene analysis service platform 100.

The controller 160 may authenticate at least one of one or more owners who provide genome, one or more users who use information on genome of the owners, and one or more managers who manage the platform through the authentication module 120.

When the user or the manager inquires about or uses information on genome of a particular owner, the controller 160 may receive particular owner's consent through the communication module 110.

The controller 160 may encrypt partial information generated by encrypting at least one piece of DNA sequence information which represents a genome-wide region, gender maker information, personal identification information, and DNA sequence information which may be related to getting insurance, and whole personal DNA sequence information together and store the encrypted information in the storage unit 150. For example, the controller 160 may encrypt the information generated by individually encrypting the four pieces of information and the whole DNA sequence information of individuals including the individually encrypted information and store the encrypted information in the storage unit 150.

The controller 160 may control the analysis module 140 to predict a personal disease risk on the basis of the phenotypic information including at least one piece of lifestyle information, information on a medical history, and clinical information, and the information on genome.

The controller 160 may provide at least one piece of information on meals, information on exercise, information on health, and information on sleep to the individual in a personalized manner on the basis of the predicted personal disease risk.

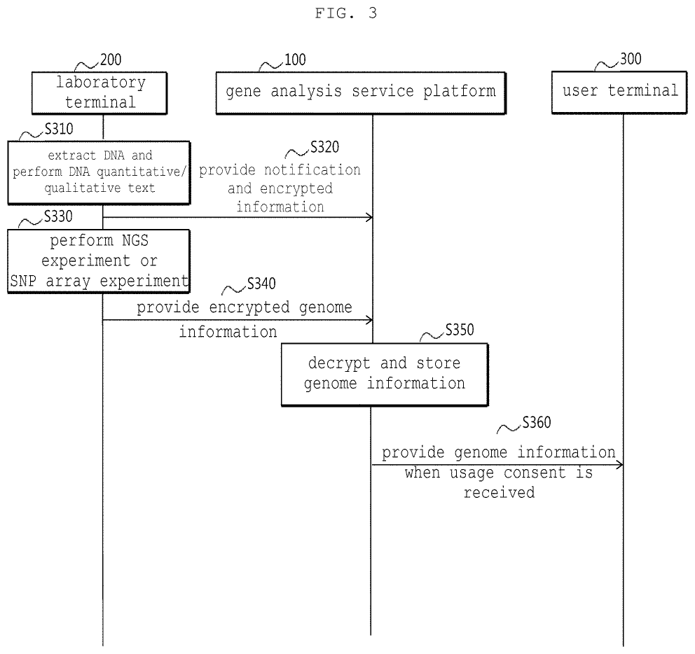

Hereinafter, various driving of the gene analysis service platform 100 according to an embodiment of the present disclosure will be described with reference to FIGS. 3 to 6.

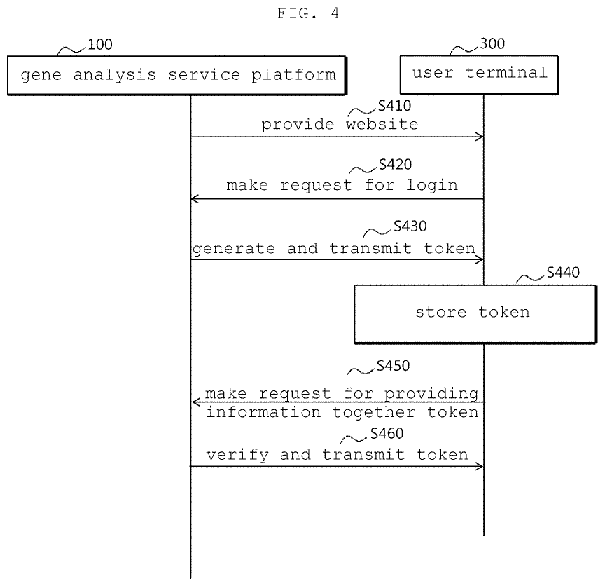

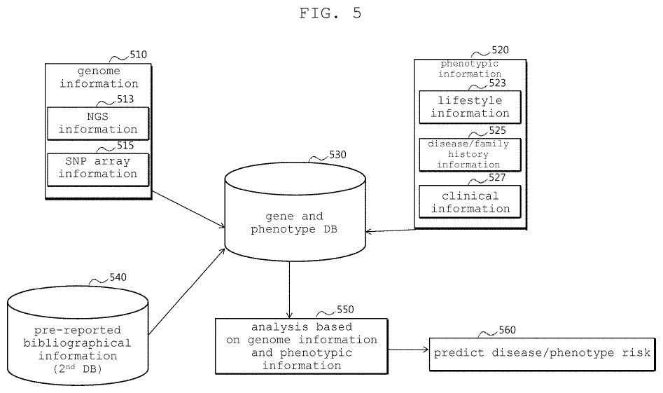

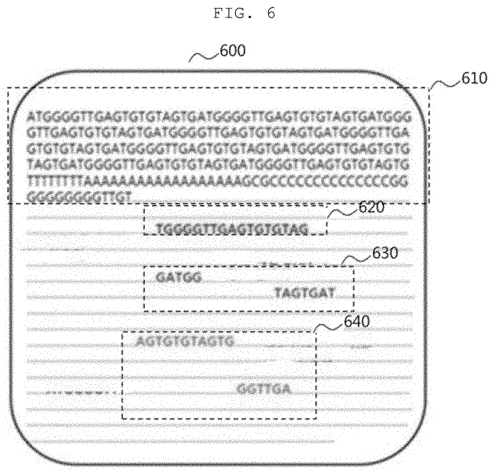

FIG. 3 is a sequence diagram illustrating driving of the gene analysis service platform 100 which monitors a process of decoding genome information according to an embodiment of the present disclosure. FIG. 4 is a sequence diagram illustrating driving of the gene analysis service platform 100 which performs a token-based authentication with the user terminal according to an embodiment of the present disclosure. FIG. 5 is a sequence diagram illustrating driving of the gene analysis service platform 100 which performs various analyses on the basis of genome information and phenotypic information according to an embodiment of the present disclosure. FIG. 6 illustrates storage of gene information in the storage unit by the gene analysis service platform 100 according to an embodiment of the present disclosure.

Referring first to FIG. 3, when a decoding and analysis service is applied for the gene analysis service platform 100, the laboratory terminal 200 extracts DNA and performs a DNA quantitative/qualitative test in S310.

A main factor that determines whether DNA sequencing data is successfully produced includes a concentration of sample DNA. Fluorometry may be applied to the specific measurement of double-stranded DNA concentrations. Although varying depending on the analysis techniques used, such as next generation sequencing (NGS), genome-wide SNP array, etc., the amount of DNA necessary for analysis is at least 200 ng for each sample. Measurements are used for reference. Using a genome-wide SNP array chip or a next generation sequencing (NGS) technique, high-throughput genome sequencing is performed on the DNA that has passed qualitative/quantitative standards. Sequencing analysis allows the acquirement of human variant information such as single nucleotide polymorphism (SNP), short indels, copy number variation, and the like.

Next, the laboratory terminal carries out an NGS test or an SNP array test in step S330. If present, a better test for extracting DNA traits of individuals may be applied to data production.

From the data produced with the NGS technique, human variant information can be secured through read filter, read mapping, variant calling, annotation analysis, etc. High-throughput integrated SNP data produced by means of genome-wide SNP array may be subjected to an algorithm suitable for calling genotypes (AA, AB, and BB) from the image source data of microarray. The genotype of a sample is determined with already secured, preexisting data serving as a seed. Genotyping of only SNP that has sufficiently been verified may decrease an error rate of clustering for calling genotyes.

The gene analysis service platform 100 receives encrypted genome information from the laboratory terminal 200 in S340, and decrypts the genome information and stores the decrypted genome information in S350.

The gene analysis service platform 100 may decrypt genome information encrypted with a public key through an RSA algorithm, store the genome information in the NAS, and store a hash value and barcode information in a cloud Relation Database Service (RDS). The gene analysis service platform 100 may manage a genome data hash value whenever genome is generated/changed/deleted, and prevent forgery and alteration.

The gene analysis service platform 100 provides genome information to the user terminal 200 which has received an owner's usage consent in S360. In this case, the gene analysis service platform 100 may authenticate the user terminal 200 through token-based authentication.

That is, the gene analysis service platform 100 may monitor a first step of extracting DNA from the genome provided from the owner, a second step of checking DNA concentration, and a third step of generating human mutant information through a gene experiment, and may decrypt, with a private key, human mutant information encrypted with a public key.

The human mutant information may include at least one piece of Single Nucleotide Polymorphism (SNP) information, Short Indel information, and copy number variation information.

The gene analysis service platform 100 may generate a personal report through personal authentication, analyze cohort gene data, and provide the analyzed gene data to an authenticated user.

FIG. 4 is a sequence diagram illustrating driving of the gene analysis service platform 100 which performs a token-based authentication with the user terminal according to an embodiment of the present disclosure.

A method by which the user terminal 300 receives a token from the gene analysis service platform 100 and use the same will be described with reference to FIG. 4.

First, the gene analysis service platform 100 provides a website in S410 and receives a login request from the user terminal 200 in S420.

The gene analysis service platform 100 generates a token and responds to the user terminal 200 in S430, and the user terminal 200 stores the token in S440.

When the user terminal 200 makes a request for information to the gene analysis service platform 100 using the stored token in S450, the gene analysis service platform 100 verifies the token and responds to the user terminal in S460.

Meanwhile, the gene analysis service platform 100 may use a cloud-based micro service for personal information security. The micro service is the term referring to service-oriented architecture and corresponds to an independent service capable of splitting one large application into several small applications to change and combine the applications. The service is small and is very loosely combined, and focuses on one function of the application. Further, each service may use a different technology stack. It is better to configure applications by smaller code bases independently managed by small teams rather than for all developer to handle one code base which involves risk in management due to a too large size thereof.

The code base is only dependent on an Application Programming Interface (API) and such architecture style is becoming popular in huge web companies such as Netflix, eBay, and Amazon. When it is required to rapidly develop a new function using the existing monolith architecture, small services may be created and each business function may be processed without making one monolith application. Services are independents and a boundary between services corresponds to a well-defined Application Programming Interface (API) which exposes a business function of the service. In the present disclosure, regions are largely divided into user/data/service provision regions and autonomy-securing services having independent expansion and functions are constructed in divided regions.

The gene analysis service platform 100 includes a cloud-based Hardware Security Module (HSM). An independent module is configured through the cloud-based micro service and personal information is encrypted and stored through management of a cloud hardware security module key. The owner may always use his/her own information, or a request for a right to use the information may be made to the owner and the information may be used for research or service through a temporary authority. Since security is managed by an individual rather than a company (research organism), data can be efficiently used and managed.

The laboratory may decode and generate genome data and process encryption from original data to protect data in a gene decoding service region, and the laboratory may support analysis of user gene and phenotypic data through uploaded data and external basis data in an analysis service region.

In a user region micro service section, subscription and authentication are managed for each of owners, managers, and users. An individual may be identified through a hash value and a barcode generated in initial subscription. In a provision region micro service section, a service for allowing an owner to read and use his/her own health information is provided and a usage right requested by a manager or a user is processed. In a banking region micro service section, a security communication section is provided for encryption technology and storage. The issue is based on the usage right token (JWT), and authentication and information exchange are processed.

FIG. 5 is a sequence diagram illustrating driving of the gene analysis service platform 100 which performs various analyses on the basis of genome information and phenotypic information according to an embodiment of the present disclosure.

Referring to FIG. 5, the gene analysis service platform 100 may store genome information 510 and phenotypic information 520 in a gene and phenotypic DB 530.

The genome information 510 includes NGS information 513. In embodiments, the NGS information 513 may be processed by fragmenting DNA and RNA sequences, may apply both whole genome sequencing of decoding the whole genome and targeted sequencing of decoding only a desired part, and may determine a paired read depth within a range from 30× to 1000× according to a read region.

There are platforms, such as Roche454, Illumina, and PacBio, for Next Generation Sequencing (NGS) and platforms, such as MiSeq, and Ion Torrent, are recently used for efficient sequencing of a target region. Panels constructed to cover a sequence within a determined target region with high accuracy can be used for sequencing, thus finding applications in the diagnosis of cancer, rare diseases, etc. For humans, a reference sequence has already been established. Thus, analysis can be carried out using a resequencing method in which the produced sequence data is compared with the reference sequence. The reads are mapped on the reference sequence and subjected to variant calling and annotation analysis to yield personal genome information. NGS data standardizes genetic variation information in vcf format.

The phenotypic information 520 may include lifestyle information 523, disease/family history information 525, and clinical information 527. The lifestyle information 523 may include information on the amount of activity, the amount of sleep, overeating, obesity, and caffeine intake, and may include various pieces of lifestyle information according to an embodiment. The disease/family history information 525 may be collected through hospitals or surveys. The clinical information may be collected through disease diagnosis, prescription of medication, and biometry.

The gene analysis service platform 100 may process a database such as GWAS catalog, OMIM, and dbGap in order to collect pre-reported bibliographic information (2^{nd} DB) and perform quality control of diseases of variation/phenotypic information of a trait, so as to create a database.

The gene analysis service platform 100 performs analysis on the basis of genome information and phenotype and predicts personal disease/phenotypic risk in S560.

The gene analysis service platform 100 may provide at least piece of information on meals, information on exercise, information on health, and information on sleep to individuals in a personalized manner on the basis of the predicted personal disease risk.

Further, the gene analysis service platform 100 may identify whether clinical gender information of a subject matches gender information generated from X and Y chromosome information of genome information and primarily determine sample confusion of the subject.

FIG. 6 illustrates storage of gene information in the storage unit by the gene analysis service platform 100 according to an embodiment of the present disclosure. The storage unit 150 may include a Network Access Server (NAS), and the gene information may be encrypted and stored in the storage unit 150. The encrypted gene information may be decrypted when personal gene analysis is performed or a user outputs personal gene information.

When personal gene information is encrypted, the controller 160 may primarily encrypt (column) DNA sequence information for identifying individuals and secondarily encrypt (file) all pieces of personal gene information in order to reinforce security.

Referring to FIG. 6, the gene analysis service platform 100 may store personal genome information 610 and DNA sequence information 620 which represents a genome-wide region. A haplotype combination may not be formed because of short distances between SNPs or correlation within particular populations, and linkage disequilibrium (LD) blocks thus formed may be transferred to the next generation. In this regard, SNP sequence information representative of LD blocks can be secured by DNA pruning analysis such that DNA sequences representative of whole genome regions can be searched for with respect to total DNA sequence information of individual populations (e.g., Asia, Europe, Africa, etc.). The corresponding sequences can be encrypted.

To this end, correlation (r2) can be calculated according to the frequency of minor alleles (alleles existing at fewer ratios in a particular population) in an SNP genotype and the value may be set to be 0.2 or 0.8 according to population data. The sequence thus selected which is representative of a whole region can be encrypted.

If suitable, a corresponding method may determine about 50,000-100,000 SNPs representative of the whole genome. Samples can be compared in pair between corresponding sequences with respect to each SNP to calculate genetic distance. For this, the IBS (Identity-By-State) method may be used. As explained in the following table, scores may be established to give two points for coincidence of both of two DNA sequences of SNP genotype between different samples, one point for coincidence of either of two DNA sequences of SNP genotype between different samples, and zero points for coincidence of none of two DNA sequences of SNP genotype between different samples.

| Samp1e 1 | Sample 2 | Score |
|---|---|---|
| A/C | A/C | 2 |
| A/T | A/A | 1 |
| A/A | A/G | 1 |
| A/A | G/G | 0 |
| G/C | G/C | 2 |

Using such a method, samples are given scores in pairs over all SNPs. An identity of 80% or higher accounts for the likelihood that the samples be derived from relatives such as parents, brothers, sisters, and the like. With an identity of 99% or higher, the samples are DNA from twins or the same person. Hence, because the analysis can prove family relations, the corresponding sequences may be encrypted.

In addition, the gene analysis service platform 100 may further store sex marker information 630. The gene analysis service platform 100 uses the sex marker to determine whether the sex on clinical information coincides with that given to the sex marker in order to prevent the samples from being indiscriminated. Furthermore, the gene analysis service platform 100 encrypts sex identification DNA sequences to make security enhancement in sex information. A sex identification SNP marker may be exemplified by rs2032678 or rs5911500, but a different marker may be applied upon execution.

Moreover, the gene analysis service platform 100 encrypts personal identification DNA information 640 which is used to prevent samples from being indiscriminated during a DNA decoding process, thereby reinforcing the security of personal DNA sequence information.

About 50 SNPs with a minor allele frequency of 5% or higher are randomly selected from a group of 50,000-100,000 SNPs representative of a whole genome and may be determined to be markers for identifying a person. To the SNPs may be added SNPs of ABO genes and markers determining RH⁺ and RH⁻. In addition, SNP information accounting for personal traits including hair thickness, eye color, earwax type, stature, eyebrow shape, length difference between the big toe and other toes, dermal senescence, acetaldehyde dehydrogenase, skin elasticity, skin color, addiction tendency, learning ability, body weight, wine preference, taste, etc. may be added. Encryption of such personal identification DNA information useful for preventing the incriminative recognition of samples during a DNA decoding process may contribute to enhancing the security of personal DNA sequence information.

The gene analysis service platform 100 may further store DNA information which may be associated with getting insurance. The gene analysis service platform 100 may encrypt DNA sequence information of SNP which may indicate risk of main cancers such as breast cancer, cancer of the digestive system (stomach cancer and colorectal cancer), a brain tumor, and pancreatic cancer, and may encrypt DNA sequence information of SNP which may indicate risk of cavity, dementia, diabetes, high blood pressure, hyperlipidemia, cardiovascular disorders, and a stoke.

The SNP may be selected on the basis of bibliographical information of a common data base such as NCBI or may be selected by analyzing a genotype and a phenotype through correlation analysis, deep-learning, and machine-learning methods of various genomes and clinical data. Through the encryption of the corresponding maker, it is possible to prevent data from being leaked to an insurance company and thus a DNA sequence owner from being discriminated.

As described above, the gene analysis service platform 100 may encrypt and store partial information generated by encrypting at least one piece of DNA sequence information which represents a genome-wide region, gender maker information, personal identification information, and DNA sequence information which may be related to getting insurance, and whole DNA sequence information of individuals together. In this case, useful personal identification information may be separately encrypted as well as the whole DNA sequence information and thus personal information protection may further increase. Further, the gene analysis service platform 100 may bi-directionally perform encryption and decryption on genome-wide genetic information, perform one-way encryption on four regions without decryption, and perform comparative analysis with a hash value in an encrypted state when a service report is generated, thereby further improving security.

In addition, the gene analysis service platform 100 may configure the phenotypic information on and the information on genome as an input matrix, extract features through a plurality of layers, and express personal disease risk in a binary number on the basis of a full connection of the extracted features. That is, it is possible to deduce the result, which is the disease risk, through learning based on a deep-learning algorithm.

The implementations of the functional operations and subject matter described in the present disclosure may be realized by a digital electronic circuit, by the structure described in the present disclosure, and the equivalent including computer software, firmware, or hardware including, or by a combination of one or more thereof. Implementations of the subject matter described in the specification may be implemented in one or more computer program products, that is, one or more modules related to a computer program command encoded on a tangible program storage medium to control an operation of a processing system or the execution by the operation.

A computer-readable medium may be a machine-readable storage device, a machine-readable storage substrate, a memory device, a composition of materials influencing a machine-readable radio wave signal, or a combination of one or more thereof.

In the description, the term “platform” or “system” is intended to encompass all kinds of mechanisms, devices, and machines for processing data, including, for example, a programmable processor, a computer, or a multiprocessor. The processing system may include, in addition to hardware, a code that creates an execution environment for a computer program when requested, such as a code that constitutes processor firmware, a protocol stack, a database management system, an operating system, or a combination of one or more thereof.

A computer program (also known as a program, software, software application, script, or code) can be written in any form of programming language, including compiled or interpreted languages, declarative or procedural languages, and it can be deployed in any form, including as a stand-alone program or module, a component, subroutine, or another unit suitable for use in a computer environment. A computer program may, but need not, correspond to a file in a file system. A program can be stored in a single file provided to the requested program, in multiple coordinated files (for example, files that store one or more modules, sub-programs, or portions of code), or in a portion of a file that holds other programs or data (for example, one or more scripts stored in a markup language document). A computer program can be deployed to be executed on one computer or on multiple computers that are located at one site or distributed across a plurality of sites and interconnected by a communication network.

A computer-readable medium suitable for storing a computer program command and data includes all types of non-volatile memories, media, and memory devices, for example, a semiconductor memory device such as an EPROM, an EEPROM, and a flash memory device, and a magnetic disk such as an external hard disk or an external disk, a magneto-optical disk, a CD-ROM, and a DVD-ROM disk. A processor and a memory may be added by a special purpose logic circuit or integrated into the logic circuit.

The implementations of the subject matter described in the specification may be implemented in a calculation system including a back-end component such as a data server, a middleware component such as an application server, a front-end component such as a client computer having a web browser or a graphic user interface which can interact with the implementations of the subject matter described in the specification by the user, or all combinations of one or more of the back-end, middleware, and front-end components. The components of the system can be mutually connected by any type of digital data communication such as a communication network or a medium.

While the specification contains many specific implementation details, these should not be construed as limitations to the scope of any disclosure or of what may be claimed, but rather as descriptions of features that may be specific to particular embodiments of particular disclosures. Certain features that are described in the specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In addition, in the specification, the operations are illustrated in a specific sequence in the drawings, but it should be understood that the operations are not necessarily performed in the shown specific sequence or that all shown operations are necessarily performed in order to obtain a preferable result. In a specific case, multitasking and parallel processing may be preferable. Furthermore, it should not be understood that a separation of the various system components of the above-mentioned implementation is required in all implementations. In addition, it should be understood that the described program components and systems usually may be integrated in a single software package or may be packaged in a multi-software product.

As described above, specific terms disclosed in the specification do not intend to limit the present disclosure. Therefore, while the present disclosure was described in detail with reference to the above-mentioned examples, a person skilled in the art may modify, change, and transform some parts without departing a scope of the present disclosure. The scope of the present disclosure is defined by the appended claims to be described later, rather than the detailed description. Accordingly, it will be appreciated that all modifications or variations derived from the meaning and scope of the appended claims and their equivalents are included in the range of the present disclosure.

What is claimed is:

1. A gene analysis service method performed by a processor, the gene analysis service method comprising:
monitoring generation of information on a genome corresponding to an individual;
encrypting and storing the generated information on the genome; and
performing analysis, based on the information on the genome and personal phenotypic information.

2. The gene analysis service method of claim 1, further comprising authenticating at least one of one or more owners who provide genomes, one or more users who use information on the genomes of the owners, and one or more managers who manage a platform through a token method.

3. The gene analysis service method of claim 2, further comprising, when the users or the managers inquire about or use information on a genome of a particular owner, receiving consent from the particular owner.

4. The gene analysis service method of claim 1, wherein the monitoring of the generation comprises monitoring a first step of extracting DNA from a genome provided from an owner, a second step of checking DNA concentration, and a third step of generating human mutant information through a gene experiment, the gene analysis service method further comprising decrypting, with a private key, human mutant information encrypted with a public key.

5. The gene analysis service method of claim 4, wherein the human mutant information includes at least one piece of Single Nucleotide Polymorphism (SNP) information, Short Indel information, and copy number variation information.

6. The gene analysis service method of claim 1, wherein the storing of the generated information comprises encrypting and storing partial information generated by encrypting at least one piece of DNA sequence information which represents a genome-wide region, gender maker information, personal identification information, and DNA sequence information related to getting insurance, and whole personal DNA sequence information together.

7. The gene analysis service method of claim 1, wherein the performing of the analysis comprises predicting personal disease risk, based on phenotypic information including at least one piece of information on a lifestyle, information on a disease history, and clinic information, and the information on the genome.

8. The gene analysis service method of claim 7, further comprising providing at least one piece of information on meals, information on exercise, information on health, and information on sleep to the individual in a personalized manner, based on the predicted personal disease risk.

9. The gene analysis service method of claim 7, wherein the predicting of the personal disease risk comprises:
configuring the phenotypic information and the information on the genome in an input matrix and extracting features through a plurality of layers; and
expressing the personal disease risk in a binary number, based on a full connection scheme of the extracted features.

10. A cloud-based gene analysis service platform comprising:
a communication module configured to communicate with a terminal connected to a laboratory;
a controller configured to monitor generation of information on a genome corresponding to an individual through the communication module; and
a storage unit configured to encrypt and store the generated information on the genome,
wherein the controller is configured to perform analysis, based on the information on the genome and personal phenotypic information.

11. The cloud-based gene analysis service platform of claim 10, further comprising an authentication module configured to perform authentication in a token method, wherein the controller is configured to authenticate at least one of one or more owners who provide genomes, one or more users who use information on the genomes of the owners, and one or more managers who manage the platform through the authentication module.

12. The cloud-based gene analysis service platform of claim 11, wherein, when the users or the managers inquire about or use information on a genome of a particular owner, the controller is configured to receive consent from the particular owner through the communication module.

13. The cloud-based gene analysis service platform of claim 10, further comprising a storage unit configured to store data, wherein the controller is configured to encrypt partial information generated by encrypting at least one piece of DNA sequence information which represents a genome-wide region, gender maker information, personal identification information, and DNA sequence information related to getting insurance, and whole personal DNA sequence information together and stores the encrypted information in the storage unit.

14. The cloud-based gene analysis service platform of claim 10, further comprising an analysis module configured to analyze gene information, wherein the controller is configured to control the analysis module to predict personal disease risk, based on phenotypic information including at least one piece of information on a lifestyle, information on a disease history, and clinic information, and the information on the genome.

15. The cloud-based gene analysis service platform of claim 14, wherein the controller is configured to provide at least one piece of information on meals, information on exercise, information on health, and information on sleep to the individual in a personalized manner, based on the predicted personal disease risk.
